(19) United States
(12) Patent Application Publication
(10) Pub. No.: US 2016/0199030 A1
(43) Pub. Date: Jul. 14, 2016

(54) DUAL MODE CMUT TRANSDUCER

(71) Applicant: KONINKLIJKE PHILIPS N.V., Eindhoven (NL)

(72) Inventors: ABHAY VIJAY PATIL, EINDHOVEN (NL); JUNHO SONG, EINDHOVEN (NL)

(73) Assignee: KONINKLIJKE PHILIPS N.V., Eindhoven (NL)

(21) Appl. No.: 14/914,007

(22) PCT Filed: Aug. 14, 2014
(86) PCT No.: PCT/EP2014/067400

§ 371 Date: Feb. 24, 2016

Related U.S. Application Data
(60) Provisional application No. 61/870,276, filed on Aug. 27, 2013.

(30) Foreign Application Priority Data
Oct. 3, 2013 (EP) 13187234.3

(51) Int. Cl. A61B 8/00; H02N 1/08; H02N 1/00

(52) U.S. Cl. CPC A61B 8/4494; H02N 1/006; H02N 1/08

(57) ABSTRACT

An ultrasonic diagnostic imaging system comprises a CMUT transducer probe with an array (10′) of CMUT cells either of the same or variable diameters operated in a conventional mode during ultrasonic signal reception and a collapsed mode during ultrasonic signal transmission. The frequency response to the CMUT cells is tailored for different clinical applications or continuously varied during echo reception by decreasing the DC bias voltage for the CMUT cells for lower frequency clinical applications, increasing the DC bias voltage for higher frequency clinical applications, or continuously decreasing the DC bias voltage as echoes are received to track the information frequency composition of the returning echo signals.

### FIELD OF THE INVENTION

This invention relates to an ultrasonic diagnostic imaging system with a CMUT transducer probe comprising an array comprising one or a plurality of CMUT cells, wherein each CMUT cell has a cell membrane, a membrane electrode, a cell floor, a substrate, and a substrate electrode; and a source of DC bias voltage coupled to the membrane electrode and the substrate electrode. Further this invention relates to a method of operating the ultrasonic diagnostic imaging system.

### BACKGROUND OF THE INVENTION

The ultrasonic transducers used for medical imaging have numerous characteristics that lead to the production of high quality diagnostic images. Among these are broad bandwidth and high sensitivity to low level acoustic signals at ultrasonic frequencies. Conventionally the piezoelectric materials which possess these characteristics have been made of PZT and PVDF materials, with PZT being the most preferred. However the ceramic PZT materials require manufacturing processes including dicing, matching layer bonding, fillers, electroplating and interconnections that are distinctly different and complex and require extensive handling, all of which can result in transducer stack unit yields that are lower than desired. Furthermore, this manufacturing complexity increases the cost of the final transducer probe. As ultrasound system mainframes have become smaller and dominated by field programmable gate arrays (FPGAs) and software for much of the signal processing functionality, the cost of system mainframes has dropped with the size of the systems. Ultrasound systems are now available in inexpensive portable, desktop and handheld form. As a result, the cost of the transducer probe is an ever-increasing percentage of the overall cost of the system, an increase which has been accelerated by the advent of higher element-count arrays used for 3D imaging. The probes used for electronic 3D imaging rely on specialized semiconductor devices application-specific integrated circuits (ASICs) which perform microbeamforming for two-dimensional (2D) arrays of transducer elements. Accordingly it is desirable to be able to manufacture transducer arrays with improved yields and at lower cost to facilitate the need for low-cost ultrasound systems, and preferably by manufacturing processes compatible with semiconductor production.

Recent developments have led to the prospect that medical ultrasound transducers can be manufactured by semiconductor processes. Desirably these processes should be the same ones used to produce the ASIC circuitry needed by an ultrasound probe such as a CMOS process. These developments have produced micromachined ultrasonic transducers or MUTs, the preferred form being the capacitive MUT (CMUT). CMUT transducers are tiny diaphragm-like devices with electrodes that convert the sound vibration of a received ultrasound signal into a modulated capacitance. For transmission the capacitive charge applied to the electrodes is modulated to vibrate/move the diaphragm of the device and thereby transmit a sound wave. Since these devices are manufactured by semiconductor processes the devices generally have dimensions in the 10-200 micron range, but can range up to device diameters of 300-500 microns. Many such individual CMUTs can be connected together and operated in unison as a single transducer element. For example, four to sixteen CMUTs can be coupled together to function in unison as a single transducer element. A typical 2D transducer array can have 2000-3000 piezoelectric transducer elements. When fabricated as a CMUT array, over one million CMUT cells will be used. Surprisingly, early results have indicated that the yields on semiconductor fab CMUT arrays of this size should be markedly improved over the yields for PZT arrays of several thousand transducer elements.

CMUTs are conventionally produced with an electrode-bearing membrane or diaphragm suspended over a substrate base carrying an opposing electrode. Referring to FIG. 9, a typical CMUT transducer cell 110 is shown in cross-section. The CMUT transducer cell 110 is fabricated along with a plurality of similar adjacent cells on a substrate 112 such as silicon. A diaphragm or membrane 114 which may be made of silicon nitride is supported above the substrate by an insulating support 116 which may be made of silicon oxide or silicon nitride. The cavity 118 between the membrane and the substrate may be air or gas-filled or wholly or partially evacuated. A conductive film or layer 120 such as gold forms an electrode on the diaphragm, and a similar film or layer 122 forms an electrode on the substrate. These two electrodes, separated by the cavity 118, form a capacitance. When an acoustic echo signal causes the membrane 114 to vibrate the variation in the capacitance can be detected, thereby transducing the acoustic wave into a corresponding electrical signal. Conversely, an a.c. signal applied to the electrodes 120, 122 causes the membrane to move and thereby transmit an acoustic signal. Due to the micron-size dimensions of a typical CMUT, numerous such CMUT cells are typically fabricated in close proximity to form a single transducer element. The individual cells can have round, rectangular, hexagonal, or other peripheral shapes.

When ultrasonic waves pass through tissue on both transmit and receive, they are affected by what is known as depth-dependent attenuation. Ultrasound is progressively attenuated the further it travels through the body and the signal to noise ratio of echoes from extended depths in the body deteriorates. This attenuation is also frequency dependent, with higher frequencies being more greatly attenuated than lower frequencies. It is for this reason that higher frequency ultrasound is used for shallow, more superficial imaging while lower frequencies are used when imaging at greater depths.

An ultrasound system describing control of the bias voltage of a conventional CMUT to vary its frequency response is known in U.S. Pat. No. 6,795,374 (Barnes et al.) In this patent Barnes et al. use a DC bias voltage to control the spacing between the diaphragm and the substrate of the conventional CMUT: the higher the bias voltage, the greater the electrostatic attraction between the diaphragm and substrate electrodes, and the closer the diaphragm is pulled toward the substrate. It is desirable to operate the CMUT with the diaphragm vibrating/moving freely above the substrate keeping the distance from the diaphragm to the substrate as close to the substrate as possible as this results in the greatest electromechanical coupling coefficient of the device; a small vibration from a returning acoustic signal will have a large effect on the variation of the capacitance of the two electrodes. This is where the CMUT is most sensitive to weak echo signals.

A disadvantage of operating the CMUT in this manner is that if the diaphragm touches the substrate it can become stuck to the floor of the CMUT cell by VanderWaals forces, rendering the CMUT inoperable. This disadvantage is recognized by Barnes et al., who suggested making the standard accommodation of the bias voltage for the expected vibration of the diaphragm, using a lower bias voltage and greater spacing between the diaphragm and substrate for strong transmission vibration of the diaphragm, and a higher bias voltage and lesser spacing when the small vibrations of echo signals are being received. In addition, they propose to augment this control with a lower bias voltage as high frequency echoes are received initially, then increase the bias voltage as echoes from deeper depths are received. This variation utilizes a phenomenon known as “spring softening”, which has an effect on the center frequency of the CMUT transducer, shifting it from a higher frequency to a lower frequency as the bias voltage is varied from a low initial voltage to a higher ending voltage during echo reception. Care must be taken to limit the high ending voltage so that VanderWaals sticking of the diaphragm is not accidentally caused. Barnes et al. are thus employing an inverse relationship between the bias voltage variation and the frequency response.

Next to the possibility of the diaphragm sticking, another drawback of the operating a conventional CMUT during the reception of an ultrasound signal is that this spring softening effect is negligible in practice, and the resultant sensitivity due to the effect is poor.

One of the ways to change the sensitivity of the CMUT transceiver is described in US2006/0004289 A1. The sensitivity of the CMUT transceiver is changed by adjusting a gap width of the CMUT. This is achieved via providing at least one element, such as protruding element or a receding element, that is formed in the cavity of the CMUT cell either on the top side of the lower electrode or at the bottom side of the diaphragm.

The disadvantage of this solution is that the adjustment of the CMUT transceiver gap has to be predefined during the manufacturing (providing either protruding element or a receding element). Thus, this adjustment remains the same throughout the CMUT's operation.

### SUMMARY OF THE INVENTION

It is an object of the present invention to provide an ultrasonic imaging system of the kind set forth in the opening paragraph which provides an improved sensitivity of the CMUT transducer over a broad range of frequencies used in ultrasonic imaging.

According to the present invention this object is achieved by providing an ultrasonic diagnostic imaging system wherein each CMUT cell of the array is arranged to operate in either of the following modes:

a conventional mode, wherein the DC bias voltage sets the CMUT membrane of the cell to vibrate freely above the cell floor during operation of the CMUT cell; and

a collapsed mode, wherein the DC bias voltage sets the CMUT membrane of the cell to be collapsed to the cell floor during operation of the CMUT cell.

The invention allows controlling the modes in which the ultrasound system is operated through setting the DC bias voltage. All cells of the CMUT array can be operated in two modes. The conventional mode of the CMUT cell operation, wherein the membrane of the cell vibrates freely above the substrate, provides the response of the CMUT cell at relative lower frequencies. The collapsed mode of the CMUT cell operation, wherein the set DC bias voltage forces the membrane to a pre-collapsed state in which the membrane is in contact with (touching) the floor, provides the response of the CMUT cell at relative higher frequencies. Variation of the DC bias voltage causes changes in the area of the membrane which is in contact with (collapsed to) the CMUT cell floor. Thus, the frequency of the CMUT's reception may be higher, compared to the conventional mode, and may be controlled. In addition to this, the collapsed mode operation provides an improved sensitivity of the system due to the closer proximity of the collapsed membrane to the cell floor.

Advantages of this invention are the possibility of using the same CMUT transducer in multi-harmonic imaging by varying frequencies of the CMUT cell's response. Moreover, the hazard of accidentally disabling the CMUT cell by VanderWaals sticking is no longer an issue, since the present invention uses this effect to its advantage.

In an embodiment of the present invention the plurality of CMUT cells includes at least one first CMUT cell and one second CMUT cell, wherein the first CMUT cell has a larger diameter than the second CMUT cell.

The difference in the diameters of the CMUT cells combined with the ultrasound system's operability in the conventional and collapsed modes may provide even further improvement in the frequency bands during at least one of transmission and reception of the ultrasound waves.

In yet another embodiment of the present invention in the conventional mode the DC bias voltage sets the membrane of the first CMUT cell to vibrate freely above the cell floor during operation of the CMUT cell; and in the collapsed mode the DC bias voltage sets the membrane of the second CMUT cell to be collapsed to the cell floor during operation of the CMUT cell.

This provides the sensitivity of the CMUT array to the relatively lower frequencies during the conventional mode of operation and relatively higher frequencies during the collapsed mode of operation.

In another embodiment of the present invention the each CMUT cell is arranged to operate in the conventional mode during transmission of an ultrasound signal and in the collapsed mode during reception of an ultrasound signal.

Transmission in the conventional mode allows maintaining tissue penetration and high frame rates for 3D imaging, for example, while reception in the collapsed mode gives control over the resolution of the imaging and reduction of the near field clutter.

In a further embodiment of the present invention the ultrasonic diagnostic system an increase in the DC bias voltage results in an increase in the center frequency of the frequency response of the CMUT cell during the operation the collapsed mode, and a decrease in the DC bias voltage results in a decrease in the center frequency of the frequency response of the CMUT cell during the operation in the collapsed mode.

When the CMUT cell is being operated in the collapsed mode the diaphragm of the cell is in contact with the floor of the cell during the operation. A DC bias voltage is controlled to vary the frequency response of the collapsed mode CMUT in a direct relationship between the bias voltage and the frequency response. As the bias voltage is decreased during echo reception, the passband of the transducer moves to progressively lower bands of frequencies. An opposite effect in frequency response can be achieved by increasing DC bias voltage. Effecting frequency control in this manner has been found to improve the sensitivity of the CMUT by an order of magnitude as compared to the frequency control techniques of the prior art.

In yet a further embodiment of the present invention each CMUT cell further comprises an area of the membrane that is collapsed to the cell floor; and an increase (decrease) in the DC bias voltage further results in an increase (decrease) of the area of the membrane that is collapsed to the cell floor.

The DC bias voltage setting defines an electrostatic force with which the membrane is being attracted towards the cell floor. Therefore, DC bias voltage increase (decrease) may result in the increase (decrease) of the area of the membrane which is in contact with the cell floor.

It is a further object of present invention to provide a method of ultrasonic imaging comprising:

selecting a frequency band for a particular clinical application;

selecting a DC bias voltage that either

sets the CMUT membrane to vibrate freely above the cell floor during the transmission of the ultrasound signal at a fundamental frequency; or

sets the CMUT membrane to be collapsed to the cell floor during the reception of the ultrasound signal; and

imaging at the fundamental frequency and/or higher harmonics of the fundamental frequency

This method may be applied in contrast agent imaging (3D low mechanical index perfusion) as higher-order ultra-harmonic (2.5fo, 3.5fo etc., wherein fo is the fundamental frequency) response of contrast agents. At lower mechanical indexes when the ultrasonic system is operated in the conventional mode, tissue does not produce higher-order harmonic response, but contrast agents do. Hence, the variable mode of operation may improve performance of the contrast agent imaging, in particular cardiac perfusion imaging. Also, having discrete modes of operation can help suppress harmonic frequencies during transmission. For example, bipolar or unipolar (non-arbitrary waveform generators) untrasoinic transducer emit higher order harmonics that can degrade the performance of harmonic imaging modes.

This method can be also used in shear wave elastrography, wherein the conventional lower frequencies mode is used for shear wave generation (providing better penetration into the tissue), and the collapsed higher frequencies mode is used for imaging (providing higher resolution). This, for example, could enhance elastographic image quality for breast, liver, prostate as well as cardiac imaging applications, where shear wave imaging has big impact.

Other possible clinical applications of the method according to the principles of the current invention may be opto-acoustic and high intensity focused ultrasound.

These and other aspects of the invention will be apparent from and elucidated with reference to the embodiments described hereinafter.

### BRIEF DESCRIPTION OF THE DRAWINGS

In the drawings:

FIG. 1 illustrates in block diagram form an ultrasonic diagnostic imaging system arranged to be operated in accordance with the principles of the present invention,

FIG. 2 illustrates a conventional CMUT cell controlled by a DC bias voltage and driven by an r.f. drive signal,

FIGS. 3a-3d illustrate the principles of collapsed mode CMUT operation applied in an implementation of the present invention,

FIG. 4 illustrates the frequency response of a collapsed mode CMUT transducer with a fixed DC bias voltage,

FIG. 5 illustrates the frequency response of a collapsed mode CMUT transducer with a DC bias voltage varied in accordance with the present invention,

FIGS. 6a and 6b illustrate the variation of the passband of a collapsed mode CMUT transducer in accordance with the present invention when varied by the PEN/GEN/RES control of an ultrasound system,

FIG. 7 illustrates the change in frequency of returning echo signals as a function of time and depth,

FIG. 8 illustrates the variation of the DC bias voltage used to respond to the changing frequencies of returning echo signals shown in FIG. 7,

FIG. 9 illustrates in cross-section a typical CMUT cell of the prior art,

FIG. 10 illustrates an embodiment of a method,

FIG. 11a illustrates an example of the spectrum data received scattered by the micro-bubbles flowing in the 200 micron thick channel,

FIG. 11b-11d illustrates ultrasound images reconstructed for 2^{nd}, 3d and 4^{th} harmonic frequencies of the received signal,

FIG. 12a illustrates the ultrasounds array operation in conventional and collapsed modes,

FIG. 12b illustrates the ultrasound array comprising CMUT cells of different diameters, and

FIG. 12c illustrates the transducers sensitivity during the conventional mode and collapsed mode of the system operation.

### DETAILED DESCRIPTION OF EMBODIMENTS

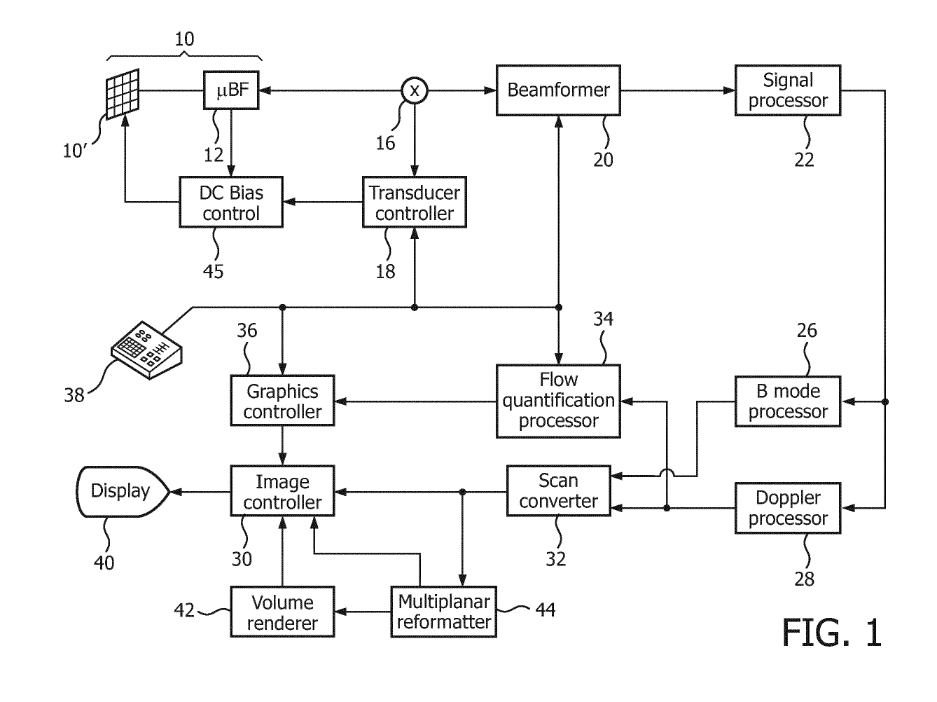

Referring first to FIG. 1, an ultrasonic diagnostic imaging system with a frequency-controlled CMUT probe is shown in block diagram form. In FIG. 1 a CMUT transducer array 10′ is provided in an ultrasound probe 10 for transmitting ultrasonic waves and receiving echo information. The transducer array 10′ is a one- or a two-dimensional array of transducer elements capable of scanning in a 2D plane or in three dimensions for 3D imaging. The transducer array is coupled to a microbeamformer 12 in the probe which controls transmission and reception of signals by the CMUT array cells. Microbeamformers are capable of at least partial beamforming of the signals received by groups or “patches” of transducer elements as described in U.S. Pat. No. 5,997,479 (Savord et al.), U.S. Pat. No. 6,013,032 (Savord), and U.S. Pat. No. 6,623,432 (Powers et al.) The microbeamformer is coupled by the probe cable to a transmit/receive (T/R) switch 16 which switches between transmission and reception and protects the main beamformer 20 from high energy transmit signals when a microbeamformer is not used and the transducer array is operated directly by the main system beamformer. The transmission of ultrasonic beams from the transducer array 10 under control of the microbeamformer 12 is directed by a transducer controller 18 coupled to the T/R switch and the main system beamformer 20, which receives input from the user's operation of the user interface or control panel 38. One of the functions controlled by the transducer controller is the direction in which beams are steered. Beams may be steered straight ahead from (orthogonal to) the transducer array, or at different angles for a wider field of view. Transducer controller 18 and microbeamformer can be coupled to the CMUT transducer array 10′ via a DC bias control 45. The DC bias control 45 sets DC bias voltage(s) that can be applied to the CMUT cells.

The partially beamformed signals produced by the microbeamformer 12 on receive are coupled to a main beamformer 20 where partially beamformed signals from individual patches of transducer elements are combined into a fully beamformed signal. For example, the main beamformer 20 may have 128 channels, each of which receives a partially beamformed signal from a patch of dozens or hundreds of CMUT transducer cells. In this way the signals received by thousands of transducer elements of a CMUT transducer array can contribute efficiently to a single beamformed signal.

The beamformed signals are coupled to a signal processor 22. The signal processor 22 can process the received echo signals in various ways, such as bandpass filtering, decimation, I and Q component separation, and harmonic signal separation which acts to separate linear and nonlinear signals so as to enable the identification of nonlinear (higher harmonics of the fundamental frequency) echo signals returned from tissue and microbubbles. The signal processor may also perform additional signal enhancement such as speckle reduction, signal compounding, and noise elimination. The bandpass filter in the signal processor can be a tracking filter as described above, with its passband sliding from a higher frequency band to a lower frequency band as echo signals are received from increasing depths, thereby rejecting the noise at higher frequencies from greater depths where these frequencies are devoid of anatomical information.

The processed signals are coupled to a B mode processor 26 and a Doppler processor 28. The B mode processor 26 employs detection of an amplitude of the received ultrasound signal for the imaging of structures in the body such as the tissue of organs and vessels in the body. B mode images of structure of the body may be formed in either the harmonic image mode or the fundamental image mode or a combination of both as described in U.S. Pat. No. 6,283,919 (Roundhill et al.) and U.S. Pat. No. 6,458,083 (Jago et al.) The Doppler processor 28 processes temporally distinct signals from tissue movement and blood flow for the detection of the motion of substances such as the flow of blood cells in the image field. The Doppler processor typically includes a wall filter with parameters which may be set to pass and/or reject echoes returned from selected types of materials in the body. For instance, the wall filter can be set to have a passband characteristic which passes signal of relatively low amplitude from higher velocity materials while rejecting relatively strong signals from lower or zero velocity material. This passband characteristic will pass signals from flowing blood while rejecting signals from nearby stationary or slowing moving objects such as the wall of the heart. An inverse characteristic would pass signals from moving tissue of the heart while rejecting blood flow signals for what is referred to as tissue Doppler imaging, detecting and depicting the motion of tissue. The Doppler processor receives and processes a sequence of temporally discrete echo signals from different points in an image field, the sequence of echoes from a particular point referred to as an ensemble. An ensemble of echoes received in rapid succession over a relatively short interval can be used to estimate the Doppler shift frequency of flowing blood, with the correspondence of the Doppler frequency to velocity indicating the blood flow velocity. An ensemble of echoes received over a longer period of time is used to estimate the velocity of slower flowing blood or slowly moving tissue.

The structural and motion signals produced by the B mode and Doppler processors are coupled to a scan converter 32 and a multiplanar reformatter 44. The scan converter arranges the echo signals in the spatial relationship from which they were received in a desired image format. For instance, the scan converter may arrange the echo signal into a two dimensional (2D) sector-shaped format, or a pyramidal three dimensional (3D) image. The scan converter can overlay a B mode structural image with colors corresponding to motion at points in the image field corresponding with their Doppler-estimated velocities to produce a color Doppler image which depicts the motion of tissue and blood flow in the image field. The multiplanar reformatter will convert echoes which are received from points in a common plane in a volumetric region of the body into an ultrasonic image of that plane, as described in U.S. Pat. No. 6,443,896 (Detmer). A volume renderer 42 converts the echo signals of a 3D data set into a projected 3D image as viewed from a given reference point as described in U.S. Pat. No. 6,530,885 (Entrekin et al.) The 2D or 3D images are coupled from the scan converter 32, multiplanar reformatter 44, and volume renderer 42 to an image processor 30 for further enhancement, buffering and temporary storage for display on an image display 40. In addition to being used for imaging, the blood flow velocity values produced by the Doppler processor 28 are coupled to a flow quantification processor 34. The flow quantification processor produces measure of different flow conditions such as the volume rate of blood flow. The flow quantification processor may receive input from the user control panel 38, such as the point in the anatomy of an image where a measurement is to be made. Output data from the flow quantification processor is coupled to a graphics processor 36 for the reproduction of measurement values with the image on the display 40. The graphics processor 36 can also generate graphic overlays for display with the ultrasound images. These graphic overlays can contain standard identifying information such as patient name, date and time of the image, imaging parameters, and the like. For these purposes the graphics processor receives input from the user interface 38, such as a typed patient name. The user interface is also coupled to the transmit controller 18 to control the generation of ultrasound signals from the transducer array 10′ and hence the images produced by the transducer array and the ultrasound system. The user interface is also coupled to the multiplanar reformatter 44 for selection and control of a display of multiple multiplanar reformatted (MPR) images which may be used to perform quantified measures in the image field of the MPR images.

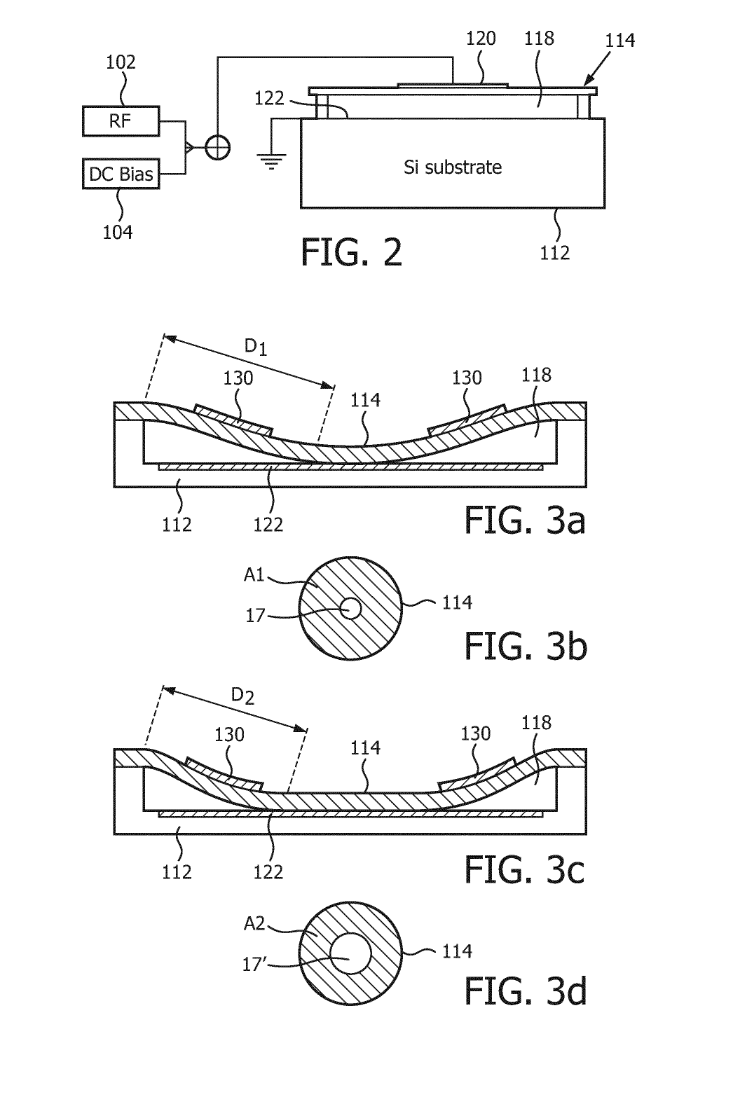

In an implementation of the present invention the elements of the transducer array 10′ comprise CMUT cells. FIG. 2 shows a conventional CMUT cell with a membrane or diaphragm 114 suspended above a silicon substrate 112 with a gap 118 therebetween. A top electrode 120 is located on the diaphragm 114 and moves with the diaphragm and a bottom electrode is located on the floor of the cell on the upper surface of the substrate 112 in this example. Other realizations of the electrode 120 design can be considered, such as electrode 120 may be embedded in the membrane 114 or it may be deposited on the membrane 114 as an additional layers. In this example, the bottom electrode 122 is circularly configured and embedded in the substrate layer 112. In addition, the membrane layer 114 is fixed relative to the top face of the substrate layer 112 and configured and dimensioned so as to define a spherical or cylindrical cavity 118 between the membrane layer 114 and the substrate layer 112.

The cell and its cavity 118 may define alternative geometries. For example, cavity 118 could define a rectangular or square cross-section, a hexagonal cross-section, an elliptical cross-section, or an irregular cross-section. Herein, reference to the diameter of the CMUT cell shall be understood as the biggest lateral dimension of the cell.

The bottom electrode 122 is typically insulated on its cavity-facing surface with an additional layer (not pictured). A preferred insulating layer is an oxide-nitride-oxide (ONO) dielectric layer formed above the substrate electrode 122 and below the membrane electrode 120. The ONO-dielectric layer advantageously reduces charge accumulation on the electrodes which leads to device instability and drift and reduction in acoustic output pressure. The fabrication of ONO-dielectric layers on a CMUT is discussed in detail in European patent application no. 08305553.3 by Klootwijk et al., filed Sep. 16, 2008 and entitled “Capacitive micromachined ultrasound transducer.” Use of the ONO-dielectric layer is desirable with pre-collapsed CMUTs, which are more susceptible to charge retention than CMUTs operated with suspended membranes. The disclosed components may be fabricated from CMOS compatible materials, e.g., Al, Ti, nitrides (e.g., silicon nitride), oxides (various grades), tetra ethyl oxysilane (TEOS), poly-silicon and the like. In a CMOS fabrication, for example, the oxide and nitride layers may be formed by chemical vapor deposition and the metallization (electrode) layer put down by a sputtering process. Suitable CMOS processes are LPCVD and PECVD, the latter having a relatively low operating temperature of less than 400° C. Exemplary techniques for producing the disclosed cavity 118 involve defining the cavity in an initial portion of the membrane layer 114 before adding a top face of the membrane layer 114. Other fabrication details may be found in U.S. Pat. No. 6,328,697 (Fraser). In the exemplary embodiment depicted in FIG. 2, the diameter of the cylindrical cavity 118 is larger than the diameter of the circularly configured electrode plate 122. Electrode 120 may have the same outer diameter as the circularly configured electrode plate 122, although such conformance is not required. Thus, in an exemplary implementation of the present invention, the membrane electrode 120 is fixed relative to the top face of the membrane layer 114 so as to align with the electrode plate 122 below. The electrodes of the CMUT provide the capacitive plates of the device and the gap 118 is the dielectric between the plates of the capacitor. When the diaphragm vibrates, the changing dimension of the dielectric gap between the plates provides a changing capacitance which is sensed as the response of the CMUT to a received acoustic echo. The spacing between the electrodes is controlled by applying a DC bias voltage 104 to the electrodes with a DC bias circuit. For transmission the electrodes 120, 122 are driven by an r.f. signal generator 102 whose a.c. signal causes the diaphragm to vibrate and transmit an acoustic signal. The DC bias voltage can be analogized to a carrier wave with the r.f. signal modulating the carrier in the transmission of the acoustic signal.

In accordance with the principles of the present invention the CMUT cell of the array 10′in FIG. 1 can be operated in one of the following modes: a conventional mode and a collapsed mode.

During the conventional mode of operation the DC bias voltage applied to the electrodes 120 and 122 104 is kept below a threshold value. This threshold value may dependent on the exact design of the CMUT cell and is defined as the DC bias voltage below which the membrane does not get stuck (contact) to the cell floor by VanderWaals force during the vibration. Thus, when the bias is set below the threshold value the membrane vibrates freely above the cell floor during operation of the CMUT cell.

The conventional mode of operation can be characterized as the mode with lower frequencies and intensities of ultrasound wave, in comparison with the collapsed mode defined below.

During the collapsed mode the DC bias voltage is operated at the values above the threshold. According to the present invention the CMUT cell is set by the DC bias voltage to a pre-collapsed state in which the membrane 114 is in contact with the floor of the cavity 118 as shown in FIG. 3a. This is accomplished by applying a DC bias voltage to the two electrodes as indicated in FIG. 2. In the illustrated collapsed mode implementation the membrane electrode 120 is formed as a ring electrode 130. Other implementations may use a continuous disk electrode which advantageously provides the pull-down force for collapse at the center of the membrane as well as peripherally. When the membrane 114 is biased to its collapsed state as shown in FIGS. 3a and 3b, the center area of the membrane is in contact with the floor of the cavity 118. As such, the center of the membrane 114 does not move during operation of the CMUT. Rather, it is the peripheral area of the membrane 114 which moves, that which is above the remaining open void of the cavity 118 and below the ring electrode. By forming the membrane electrode 130 as a ring, the charge of the upper plate of the capacitance of the device is located above the area of the CMUT which exhibits the motion and capacitive variation when the CMUT is operating as a transducer. Thus, the coupling coefficient of the CMUT transducer is improved.

As has been indicated the membrane 114 may be brought to its collapsed state in contact with the center of the floor of the cavity 118 by applying DC bias voltage above the threshold value, which is a function of the cell diameter, the gap between the membrane and the cavity floor, and the membrane materials and thickness. As the voltage is increased, the capacitance of the CMUT cell is monitored with a capacitance meter. A sudden change in the capacitance indicates that the membrane has collapsed to the floor of the cavity. The membrane can be biased downward until it just touches the floor of the cavity as indicated in FIG. 3a, or can be biased further downward as shown in FIG. 3b to increase collapse beyond that of minimal contact, such as the area of the membrane that is collapsed to the cell floor increases.

In accordance with the principles of the present invention, the frequency response of a collapsed mode CMUT is varied by adjusting the DC bias voltage applied to the CMUT electrodes after collapse. As a result, the resonant frequency of the CMUT cell increases as higher DC bias is applied to the electrodes. The principles behind this phenomenon are illustrated in FIGS. 3a-3d. The cross-sectional views of FIGS. 3a and 3c illustrate this one dimensionally by the distances D₁ and D₂ between the outer support of the membrane 114 and the point where the membrane begins to touch the floor of the cavity 118 in each illustration. It can be seen that the distance D₁ is a relatively long distance in FIG. 3a when a relatively low bias voltage is applied after collapse, and the distance D₂ in FIG. 3c is a much shorter distance when a higher bias voltage is applied. These distances can be analogized to long and short strings which are held by the ends and then plucked. The long, relaxed string will vibrate at a much lower frequency when plucked than will the shorter, tighter string. Analogously, the resonant frequency of the CMUT cell in FIG. 3a will be lower than the resonant frequency of the CMUT cell in FIG. 3c which is subject to the higher DC pulldown bias voltage.

The phenomenon can also be appreciated from the two dimensional illustrations of FIGS. 3b and 3d, as it is in actuality a function of the effective operating area of the CMUT membrane. When the membrane 114 just touches the floor of the CMUT cell as shown in FIG. 3a, the effective vibrating area A₁ of the noncontacting (free vibrating) portion of the cell membrane 114 is large as shown in FIG. 3b. The small hole in the center 17 represents the center contact region of the membrane. The large area membrane will vibrate at a relatively low frequency. This area 17 is an area of the membrane 114, which is collapsed to the floor of the CMUT cell. But when the membrane is pulled into deeper collapse by a higher bias voltage as in FIG. 3c, the greater central contact area 17′ results in a lesser free vibrating area A₂ as shown in FIG. 3d. This lesser area A₂ will vibrate at a higher frequency than the larger A₁ area. Thus, as the DC bias voltage is decreased the frequency response of the collapsed CMUT cell decreases, and when the DC bias voltage increases the frequency response of the collapsed CMUT cell increases.

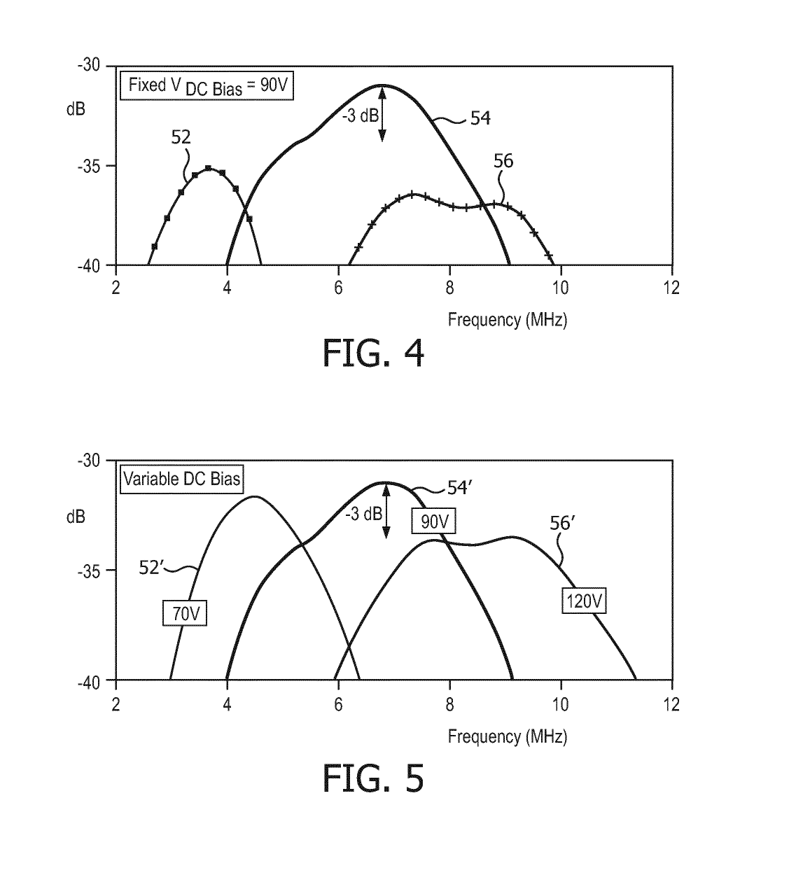

FIGS. 4 and 5 illustrate how variation of the DC bias voltage of a collapsed CMUT can optimize the transducer for a particular desired frequency of operation. FIG. 4 illustrates a frequency response curve 54 for a CMUT transducer with a fixed DC bias being operated in the collapsed mode, which has a nominal center frequency of around 6 MHz. When the transducer probe is operated with signals at 6 MHz it is seen that the response curve of signals around 6 MHz exhibits good sensitivity, as it is operating in the center of the passband. But when the probe is operated with signals at a low band such as 4 MHz, it is seen that a band 52 of signals in this range rolls off because the band 52 is at the lower end of the response curve 54 and is down around 4 dB below peak. Similarly, when operated around 8 MHz as shown by band 56, the high frequency rolloff of the transducer passband 54 attenuates signals down by 6 dB below peak. But when the DC bias voltage is varied to optimize the transducer for the desired frequency band of operation, this skirt attenuation is avoided. As FIG. 5 illustrates, when a DC bias of 70 volts is used for low band operation, 90 volts is used for mid-band operation, and 120 volts is used for high band operation in this example, the desired passbands 52′, 54′ and 56′ are in the center of the shifted resonant transducer passband in each case, resulting in little or no side skirt frequency rolloff.

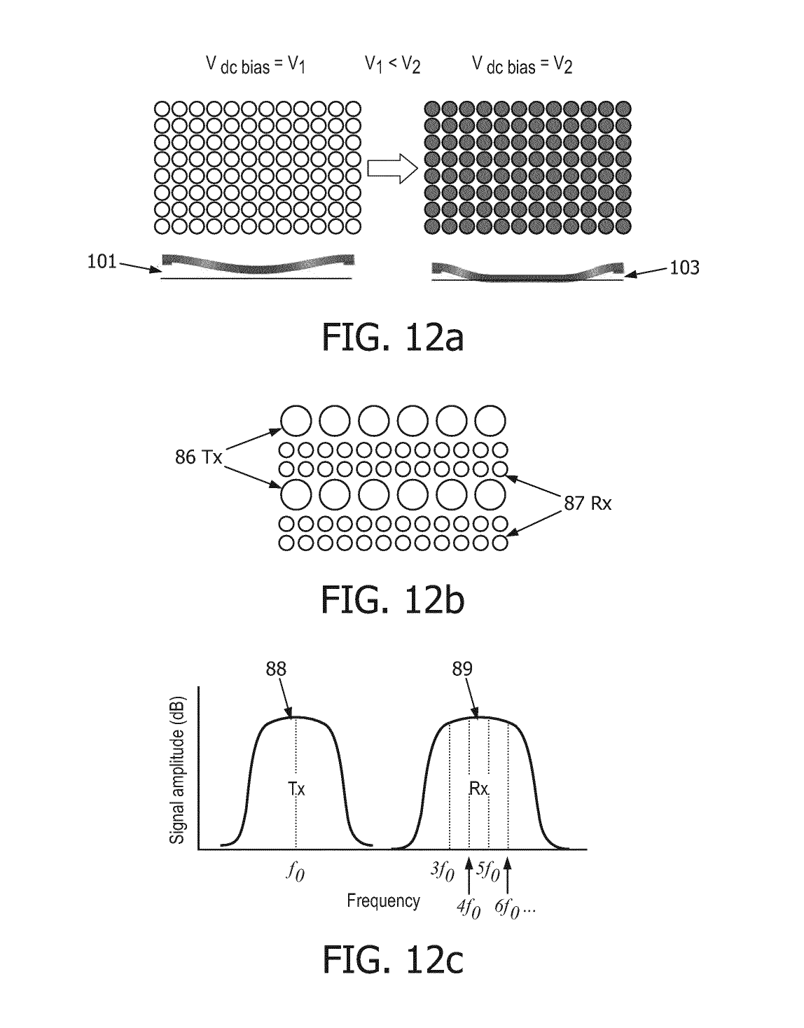

One of the examples of the ultrasound array operation is illustrated in FIG. 12a. During transmission of the ultrasound signal the CMUT cells in the array are operated in the conventional mode. In this mode the membrane 114 is set to vibrate freely above the cell floor by the DC bias voltage V₁. The cross-sectional view of the membrane 114 position in relation to the cell floor is denoted as 101. For the simplicity, other parts of the CMUT cell are not shown. During reception of the echo signals the DC bias control 45 sets the DC bias voltage to the value V₂ that is higher than both V₁ and the threshold value of the CMUT cell. Therefore, the membranes of the cells in the array are set to be collapsed to the cell floor during operation. The cross-sectional view of the membrane 114 position in relation to the cell floor in collapsed mode is denoted as 103. The resonant frequency increases as higher DC bias is applied to the electrodes in the collapsed mode. FIG. 12c illustrates a frequency response of the transducers in transmission as band 88, when lower DC bias V₁ may be used to transmit high acoustic pressure wave at low frequency (fo), and in reception as band 89, when high DC bias V₂ may be used to increase the frequency response of the cMUT transducer (3 fo, 4 fo, 5 fo, . . . ).

In accordance to the principles of the present invention the frequency sensitivity of the ultrasonic system response may be broadened even further by providing the array, wherein the CMUT cells have different diameters. Biasing of the cells of a different diameter may allow transmitting ultrasound waves at variable fundamental frequencies. CMUT cells of larger diameter have lower fundamental frequency compared to the cells of the smaller diameter.

FIG. 12b illustrates a transducer array comprising two pluralities 86,87 of the CMUT cells that are mutually different in diameters and may be located on the same substrate 112. A first plurality 86 of CMUT cell has larger diameter compared to a second plurality 87 of the CMUT cell. The plurality 86 may be operated to transmit high acoustic pressure signals at relatively low frequency (e.g. at fo between 1 and 4 MHz), while the plurality 87 of the CMUTs with smaller diameter may be operated during the reception of the high order harmonics of echo signals (3 fo, 4 fo, 5 fo, . . . ). The frequency response of the transducers having different diameters in array is schematically illustrated in FIG. 12c as well. The membranes of the CMUTs with different diameters can have various sizes and thickness in order to meet desired frequency sensitivity requirements. The array may also include cells with more than two different diameters, which may also be fabricated on different substrates and assembled in the system's array afterword.

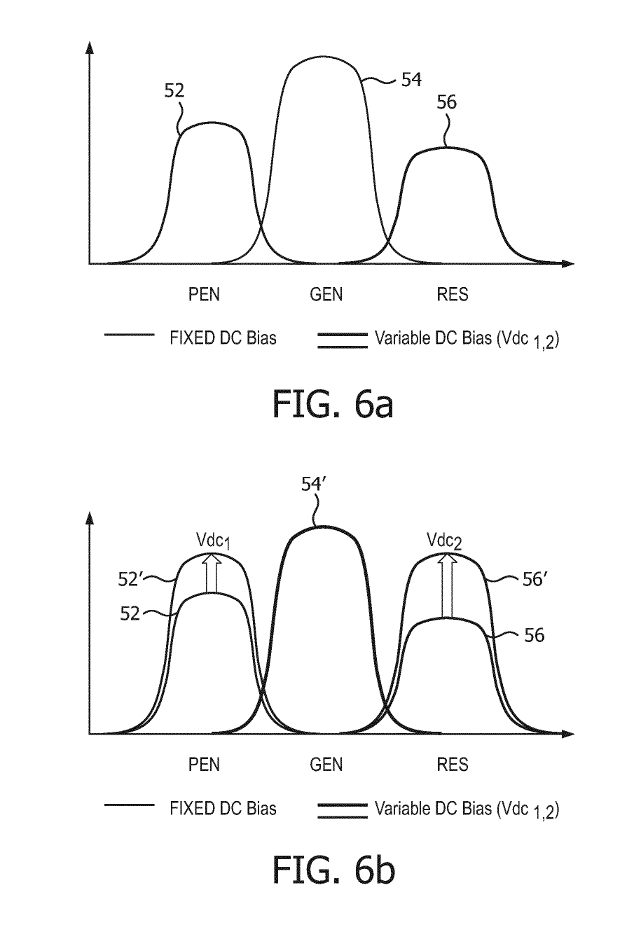

An ultrasound system generally provides the operating clinician with the ability to set the frequency band of operation for a particular clinical application. Typically, the clinician can adjust a user control on the system control panel 38 to excite the transducer at lower frequencies with a nominal center frequency below 4 MHz for better penetration (PEN mode 52), higher frequencies with a nominal center frequency between 8 and 12 MHz for better resolution (RES mode 56), or a range of intermediate frequencies with a nominal center frequency between 4 and 8 MHz for general applications requiring both good penetration and good resolution (GEN mode 54) as illustrated in FIGS. 5 and 6a. When only a single DC bias setting is used, a compromise band of CMUT transducer operation must be used for all three system settings. But with the ability to vary the CMUT transducer frequency response band in correspondence with the clinical application setting, a lower band 52′ can be used in the PEN mode, an intermediate band 54′ used in the GEN mode, and a high band 56′ used in the RES mode as shown in FIG. 6b. The PEN and RES bands 52′ and 56′ are seen to exhibit improved sensitivity as compared to the lower response of bands 52 and 56 when a fixed DC bias optimized for the center GEN band is used. Thus, the frequency response of the variable band collapsed mode CMUT transducer probe is tailored to the needs of a particular clinical application.

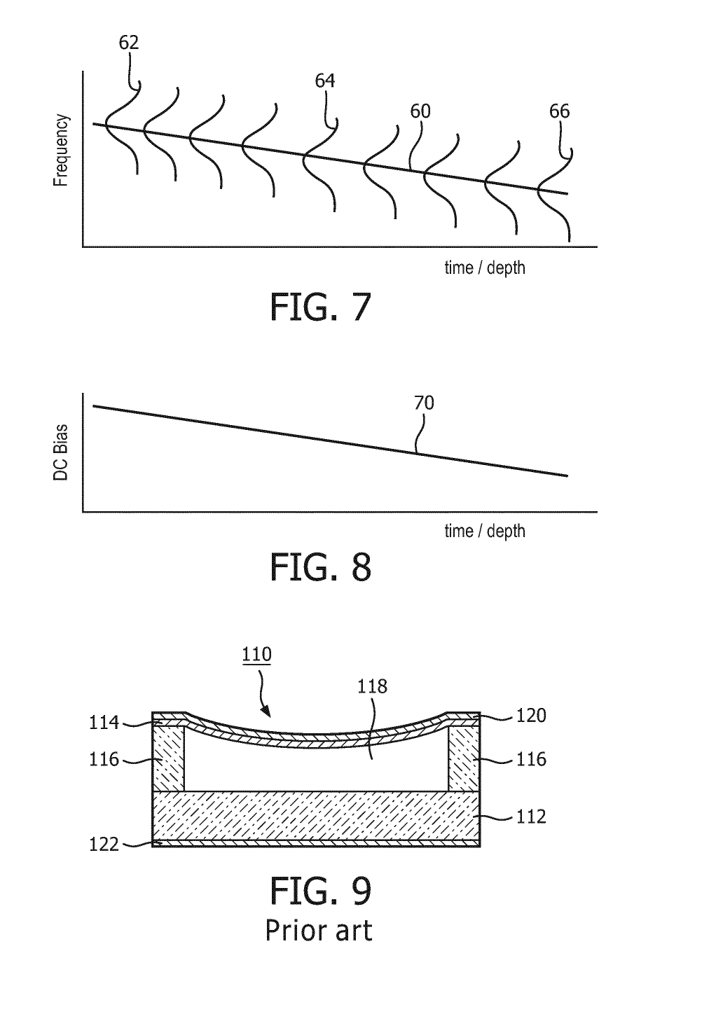

The frequency response of a variable band collapsed mode CMUT transducer can also be continually varied during echo reception, offering the same effect as a system tracking filter as shown in FIGS. 7 and 8. FIG. 7 illustrates the progressive decline in the center frequency of echo signals 62, 64, 66 as the echoes are received from increasing depths over time as shown by the ordinate axis of the illustration. The line 60 plots the steady decline in center frequency with depth (time). As echoes are received from shallow depths and then from progressively deeper depths, the DC bias voltage of a collapsed mode CMUT is varied from a higher voltage to a lower voltage as shown by the line 70 in FIG. 8, and the center frequency of the CMUT cells declines correspondingly. The frequency response of the collapsed mode CMUT array is continually tailored to follow the depth-dependent frequency attention by this method of DC bias control.

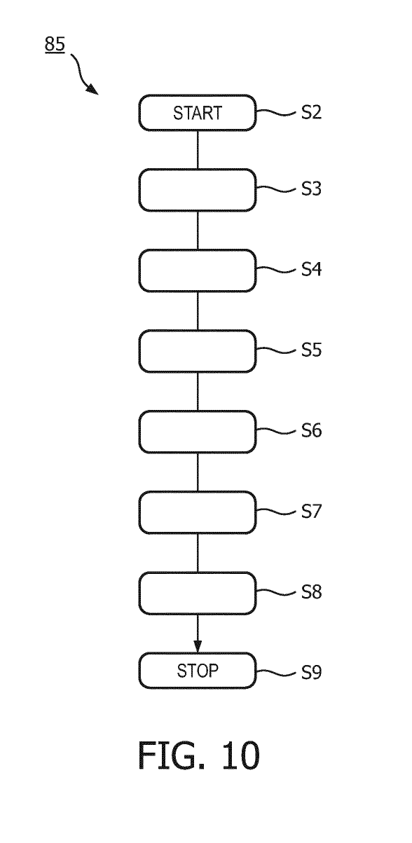

According to the principles of present invention a method 85 of ultrasonic imaging is illustrated in FIG. 10. The method starts with step S2. Then in step S3 an array comprising one or plurality CMUT cell is provided. An array may comprise CMUT cells of the same diameter or different diameters, depending on the potential clinical application of the ultrasound system. Coupling of a DC bias voltage between two CMUT electrodes: the membrane electrode and the substrate electrode is arranged in step S4. Further in step S5 the clinician can adjust a user control on the system control panel 38 to select a frequency band for a particular clinical application. The selection of the frequency band is realized by setting different DC bias voltages via the DC bias control 45. The same DC bias control may be used in step S6 in order to operate the system in the conventional mode during transmission of the ultrasound signal. During S6 step the DC bias voltage is controlled such as the CMUT cell membrane 114 is set to vibrate freely above the cell floor. The system transmits the ultrasound signal at a fundamental frequency of the CMUT cell which can be defined by the applied DC bias voltage that is kept below the threshold value. In the next step S7 reception of the ultrasound signal is performed by the system in the collapsed mode. The DC bias control 45 may be used in step S7 in order to operate the system in the collapsed mode. During S7 step the DC bias voltage is controlled such as the CMUT cell membrane 114 is set to be collapsed to the cell floor during the reception. The area of the membrane (17,17′) which is collapsed to the cell floor during the reception is determined by the frequency band of the particular clinical application that is selected in step S5. In step S8 imaging of the received echo signal at the fundamental frequency and/or higher harmonics of the fundamental frequency is performed. The method finishes in step S9.

One of the advantages of the present method is that during the transmission the system can be operated in conventional mode via biasing CMUTs of one diameter, while during the reception the system can be operated in the collapsed mode via biasing CMUTs cells of a different diameter to maximize the receiving sensitivity and keep the harmonic frequency well separated from the fundamental frequency.

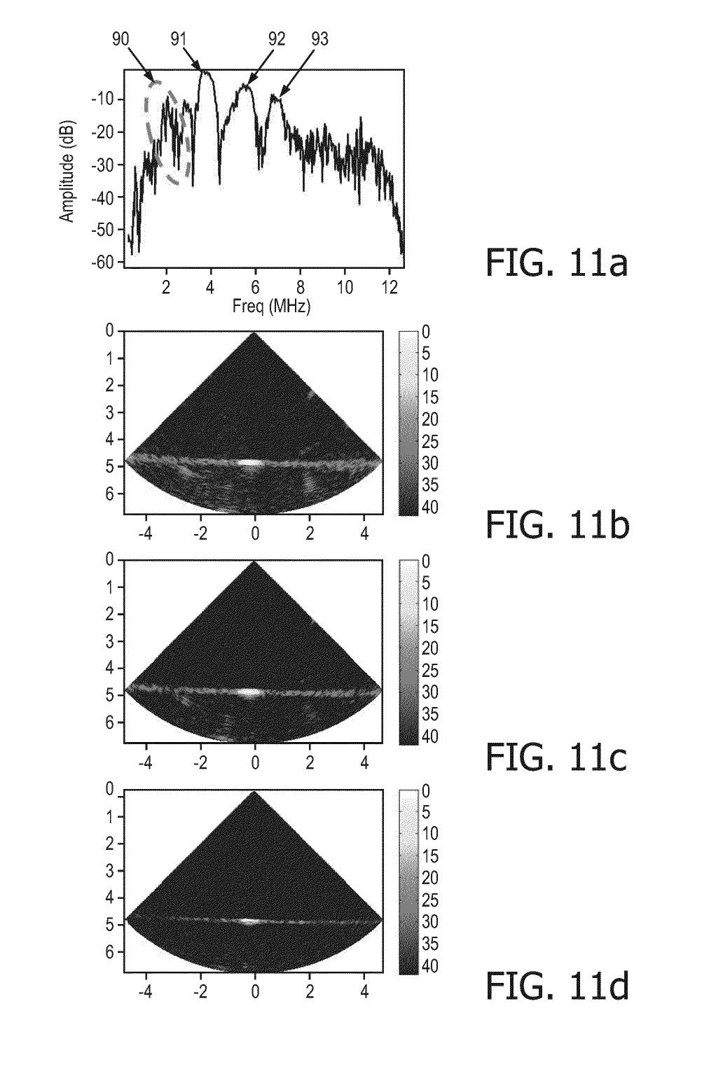

One of the examples of the particular clinical applications performed in step 83 may be a contrast agent imaging commonly applied is low mechanical index (MI) cardiac perfusion imaging. The feasibility of using higher order harmonics for imaging contrast signal is presented in FIGS. 11a, 11b, 11c and 11d. The ultrasound images were obtained using a prototype of the applicant's ultrasonic system Voyager based on the CMUT array. The cMUT cell was excited by a.c. electrical signal with an amplitude of 15 V centered at 1.5 MHz (MI˜0.1). FIG. 11a illustrates the spectra of the received ultrasound RF data sets, wherein the fundamental frequency 90, its second 91, third 92 and fourth 93 harmonics indicated. FIGS. 11b, 11c, and 11d illustrate the images re-constructed from the 2nd, 3rd and 4th order scattering from the microbubbles use as a contrast agent. The dynamic range of the images is 40 dB. In this the contrast agent imaging relatively low frequencies may reflect response from a body tissue and relatively higher frequencies may reflect response from the contrast agents.

Yet another example of the clinical application of the present invention is enhanced image dynamic elastography, specifically for cardiac applications. Dynamic elastography (e.g. shear wave imaging) uses high intensity pulses to create a mechanical shear wave in the region of interest (ROI). The speed of the wave is then tracked with alternate pulses. The local velocity estimates are used to back-calculate tissue shear modulus. In cardiac applications, the ROIs may be as deep as 15-20 cms. Hence lower frequencies are desired, especially for generating shear waves Dual mode ultrasonic system based on cMUTs (operating in the conventional and the collapsed modes) can potentially provide lower frequencies with desired intensities for shear wave generation and intermediate-higher frequencies for tracking/imaging. In the preferred embodiment the generation of the shear wave may be done through the activation of the CMUT cells of the relatively bigger diameter, while the tracking echo signals may be transmitted by the CMUT cells of the relatively smaller diameter.

Another example of the particular clinical application is opto-acoustics. Opto-acoustics is a modality that uses optical excitation to create an acoustic response from the tissue. The received acoustic response is often high-bandwidth RF response (higher than 10 MHz). Dual mode ultrasonic system based on cMUTs could help detect the range of frequencies in the received opto-acoustic response and capture the entire bandwidth.

One more example of the clinical application is high intensity focused ultrasound (HIFU) that has been established in the literature as a non-invasive approach for ablating/dissolving lesions using focused ultrasound energy.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

1. An ultrasonic diagnostic imaging system with a CMUT transducer probe comprising:
an array comprising one or a plurality of CMUT cells, wherein each CMUT cell has a cell membrane, a membrane electrode, a cell floor, a substrate, and a substrate electrode; and
a source of DC bias voltage coupled to the membrane electrode and the substrate electrode;
wherein
each CMUT cell is arranged to operate in either of the following modes:
a conventional mode, wherein the DC bias voltage sets the CMUT membrane of the cell to vibrate freely above the cell floor during operation of the CMUT cell; and
a collapsed mode, wherein the DC bias voltage sets the CMUT membrane of the cell to be collapsed to the cell floor during operation of the CMUT cell.

2. The ultrasonic diagnostic system according to claim 1, wherein the plurality of CMUT cells includes at least one first CMUT cell and one second CMUT cell, wherein the first CMUT cell has a larger diameter than the second CMUT cell.

3. The ultrasonic diagnostic system according to claim 2, wherein in the conventional mode the DC bias voltage sets the membrane of the first CMUT cell to vibrate freely above the cell floor during operation of the CMUT cell; and in the collapsed mode the DC bias voltage sets the membrane of the second CMUT cell to be collapsed to the cell floor during operation of the CMUT cell.

4. The ultrasonic diagnostic system according to claim 1, wherein each CMUT cell is arranged to operate in the conventional mode during transmission of an ultrasound signal and in the collapsed mode during reception of an ultrasound signal.

5. The ultrasonic diagnostic system according to claim 1, wherein the DC bias voltage is selectable for different clinical applications.

6. The ultrasonic diagnostic system according to claim 5 wherein an increase in the DC bias voltage results in an increase in the center frequency of the frequency response of the CMUT cell during the operation in the collapsed mode, and a decrease in the DC bias voltage results in a decrease in the center frequency of the frequency response of the CMUT cell during the operation in the collapsed mode.

7. The ultrasonic diagnostic imaging system according to claim 6, wherein the DC bias voltages for the different clinical applications are set using an ultrasound system control.

8. The ultrasonic diagnostic imaging system according to claim 7, wherein the ultrasound system control further comprises a selection of the following clinical applications: a relatively low frequency penetration operating in a frequency band with a nominal center frequency below 4 MHz, high frequency resolution operating in a frequency band with a nominal center frequency between 8 and 12 MHz and intermediate frequency operating in a frequency band with a nominal center frequency between 4 and 8 MHz.

9. The ultrasonic diagnostic system according to claim 1, wherein in collapsed mode each CMUT cell further comprises an area of the membrane that is collapsed to the cell floor; and
wherein an increase (decrease) in the DC bias voltage further results in an increase (decrease) of the area of the membrane which is collapsed to the cell floor.

10. The ultrasonic diagnostic imaging system according to claim 1, wherein each CMUT cell has a circular shape; and
wherein the membrane electrode further comprises a ring electrode.

11. The ultrasonic diagnostic imaging system according to claim 1, wherein the substrate electrode is overlaid with an insulating layer comprising the surface of the cell floor.

12. The ultrasonic diagnostic imaging system according to claim 1, wherein each CMUT cell is configured in a square or hexagonal shape.

13. The ultrasonic diagnostic imaging system according to claim 1, wherein a plurality of CMUT cells of the array are arranged to operate together as a unitary transducer array element.

14. A method of ultrasonic imaging comprising:
providing an array comprising one or a plurality of CMUT cells (S3), wherein at least two CMUT cells may mutually differ in diameter;
coupling a DC bias voltage between the membrane electrode and the substrate electrode of the CMUT cell (S4);
characterized in that the method further comprises:
selecting a frequency band for a particular clinical application (S5);
selecting a DC bias voltage that either
sets the CMUT membrane to vibrate freely above the cell floor during the transmission of the ultrasound signal at a fundamental frequency (S6); or
sets the same CMUT membrane to be collapsed to the cell floor during the reception of the ultrasound signal (S7); and
imaging at the fundamental frequency and/or higher harmonics of the fundamental frequency (S8).

15. The method ultrasonic diagnostic imaging according to claim 14, wherein the particular clinical application is one of a contrast agent imaging, enhanced image elastography, opto-acoustics or high intensity focused ultrasound.
